(19) United States
(12) Patent Application Publication
(10) Pub. No.: US 2021/0196315 A1
(43) Pub. Date: Jul. 1, 2021

(54) TROCAR WITH MOVABLE CAMERA AND BUILT-IN POSITION SENSOR

(71) Applicant: BIOSENSE WEBSTER (ISRAEL) LTD., Yokneam (IL)

(72) Inventors: Yehuda Algawi, Binyamina (IL); Assaf Govari, Haifa (IL); Ilya Sitnitsky, Nahariya (IL); Stanislav Katzir, Hadera (IL)

(21) Appl. No.: 16/729,435

(22) Filed: Dec. 29, 2019

(51) Int. Cl. A61B 17/34; A61B 1/005; G06T 7/00

(52) U.S. Cl. CPC A61B 17/3423; A61B 1/0052; A61B 2576/026; A61B 2562/0223; G06T 7/0014

(57) ABSTRACT

A trocar for insertion into an organ of a patient includes a cannula having a longitudinal axis, a camera fitted inside the cannula, and a movable element, which is coupled to the camera and is configured to be moved along the longitudinal axis of the cannula and to move the camera along the cannula.

### FIELD OF THE INVENTION

The present invention relates generally to invasive medical tools, and particularly to invasive medical tools incorporating a camera.

### BACKGROUND OF THE INVENTION

Techniques for image-guided probing of an organ of a patient were previously proposed in the patent literature. For example, U.S. Patent Application Publication 2011/0160535 describes a disposable access port for use in endoscopic procedures, including laparoscopic procedures. The access port includes a cannula with an embedded camera in communication with an external control box. In operation, a trocar is combined with the access port to facilitate insertion of the access port into an anatomical site. Prior to insertion, the camera is pushed inside the cannula, where it remains during insertion. The trocar is removed after the access port has been inserted to allow surgical instruments to access the anatomical site. During removal of the trocar, a portion of the trocar urges the camera out of the cannula, thereby allowing visualization of the anatomical site. The camera can be fixedly or adjustably mounted on the port. A camera may also be mounted on the trocar. The trocar may include irrigation and suction channels to facilitate a clear view of the anatomical site.

As another example, U.S. Patent Application Publication 2013/0282041 describes a viewing trocar assembly including a tubular body having a proximal end and a distal end, and an opening provided at the distal end, and at least one imaging device positioned on an outer wall of the distal end of the tubular body, wherein the at least one imaging device is adjacent to the outer wall of the distal end of the tubular body when in an inactivated position, and wherein the at least one imaging device is extended further away from the outer wall of the distal end of the tubular body when in an activated position than when in the inactivated position. The disclosed imaging device is maneuverable once positioned inside a patient's body, thus providing improved imaging capabilities.

### SUMMARY OF THE INVENTION

An embodiment of the present invention provides a trocar for insertion into an organ of a patient, the trocar including a cannula having a longitudinal axis, a camera fitted inside the cannula, and a movable element, which is coupled to the camera and is configured to be moved along the longitudinal axis of the cannula and to move the camera along the cannula.

In some embodiments, the movable element is configured to be slid in a channel formed at an inner wall of a cannula, parallel to the longitudinal axis, and wherein a distal end of the movable element is coupled to the camera.

In some embodiments, the trocar further includes a slide control handle coupled to a proximal end of the movable element, the slide control handle including a rotatable camera-position control knob configured to be rotated and, by being rotated, to move the movable element and the camera.

In other embodiments, the trocar further includes a position sensor, which is attached to an inner wall of a distal end of the cannula, and is configured to generate signals indicative of a position of the distal end in the organ.

In an embodiment, the position sensor is a magnetic position sensor. In another embodiment, the camera is tilted relative to the longitudinal axis, so as to have a viewing direction that captures a distal opening of the cannula.

There is additionally provided, in accordance with another embodiment of the present invention, an apparatus including a trocar and a slide control handle. The trocar is configured for insertion into an organ of a patient, and includes a cannula having a longitudinal axis, a camera fitted inside the cannula, and a movable element, which is coupled to the camera and is configured to be moved along the longitudinal axis of the cannula and to move the camera along the cannula. The slide control handle coupled to a proximal end of the movable element and configured to move the movable element and the camera.

In some embodiments, the movable element is configured to be slid in a channel formed at an inner wall of a cannula, parallel to the longitudinal axis, wherein a distal end of the movable element is coupled to the camera, and wherein the slide control handle includes a rotatable camera-position control knob configured to be rotated and, by being rotated, slide the movable element.

There is further provided, in accordance with another embodiment of the present invention, a system including a trocar and a processor. The trocar is configured for insertion into an organ of a patient, and includes a cannula having a longitudinal axis, a camera fitted inside the cannula, a movable element, which is coupled to the camera and is configured to be moved along the longitudinal axis of the cannula and to move the camera along the cannula, and a position sensor, which is attached to an inner wall of a distal end of the cannula, and is configured to generate signals indicative of a position of the distal end in the organ. The processor is configured to, using the signals generated by the position sensor, estimate the position of the distal end of the trocar in the organ.

In some embodiments, the processor is further configured to, based on the estimated position, register an image acquired by the camera with a reference medical image, and present the image acquired by the camera and the reference medical image, registered with one another, to a user.

There is furthermore provided, in accordance with another embodiment of the present invention, a method including inserting a trocar into an organ of a patient, the trocar including a cannula having a longitudinal axis, a camera fitted inside the cannula, and a movable element, which is coupled to the camera and is configured to be moved along the longitudinal axis of the cannula and to move the camera along the cannula. The slide control handle coupled to a proximal end of the movable element and configured to move the movable element and the camera. The movable element and the camera are moved using a slide control handle coupled to a proximal end of the movable element.

There is further yet provided, in accordance with another embodiment of the present invention, a method including inserting a trocar into an organ of a patient, the trocar including a cannula having a longitudinal axis, a camera fitted inside the cannula, a movable element, which is coupled to the camera and is configured to be moved along the longitudinal axis of the cannula and to move the camera along the cannula, and a position sensor, which is attached to an inner wall of a distal end of the cannula without obstructing a field of view of the camera, and is configured to generate signals indicative of a position of the distal end in the organ. Using the signals generated by the position sensor, the position of the distal end of the trocar in the organ is estimated.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a brain procedure using a surgical apparatus comprising a trocar comprising a slidable camera and a position sensor, in accordance with an embodiment of the present invention;

FIG. 2 is a schematic, pictorial illustration of the trocar applied in the brain procedure of FIG. 1, in accordance with an embodiment of the present invention; and

FIG. 3 is a flow chart that schematically illustrates a method and algorithm for achieving a best visual image from slidable camera of FIG. 1 and registering the image with a reference medical image, in accordance with an embodiment of the present invention.

### DETAILED DESCRIPTION OF EMBODIMENTS

### Overview

Some invasive medical procedures require a way to visually guide a medical probe to an organ, such as a brain, of a patient. In some invasive procedures, to insert a medical probe or other tool into the body of a patient, a trocar, which serves as a penetrating portal, is first placed in an entry location. In addition to being a portal for the probe, the trocar, which comprises a cannula, may be used for irrigation and to drain bodily fluids, as well as other fluids. Moreover, the trocar may be equipped with a camera to assist in the visual navigation of the probe to target tissue.

For example, brain procedures may require navigating a distal end of a probe inserted into the brain via a hole made in the skull. For the procedure, a trocar with a camera may be inserted to enable a physician to acquire images of a target brain tissue, and a treating probe is advanced via the trocar and visually guided to treat the target brain tissue, for example, an infected or bleeding brain tissue.

However, if the camera of the trocar is located too far distally inside the trocar, the acquired image is obscured by blood or other matter; if the camera is located too proximally inside the trocar the acquired image is of a poor quality and may show the trocar wall rather than the desired view.

Embodiments of the present invention that are described hereinafter provide a trocar that has, fitted internally to a wall of the cannula, a movable camera and a moving mechanism to move the camera parallel to a longitudinal axis of the cannula (i.e., over a range between proximal and distal positions), to a location inside the cannula from which the camera can provide a best view, such as of target tissue and/or of a treating probe.

In some embodiments, a sliding movement of the camera is provided using a channel within the cannula, within which the camera can slide using a movable element in the channel to which the camera is coupled. In some embodiments, the movable element is a flexible tube that is coupled to the camera via a sliding adapter on which the camera is mounted. The flexible tube further contains camera wiring. The flexible tube, also called hereinafter “a camera control guide,” connects the camera to an external control that is configured to cause the control guide to slide. The external control is typically fitted with an a slide control handle coupled to a proximal end of the movable element, the slide control handle including a rotatable camera-position control knob configured to be rotated and, by being rotated, to move the movable element and the camera. For example, after the trocar has been inserted, the physician can rotate the camera-position control knob to slide the movable element along its channel, which causes the camera to slide as described above. The physician is thus able to position the camera such that the camera is not obscured and can provide a good image.

In other embodiments, other types of movable elements may be used to move the camera, such as elastic elements (e.g., a spring), a piston, or shape-memory-based mechanical elements. In yet another embodiment, the movement may be realized using, for example, an electrical motor coupled to the camera to move on a rail fitted inside the cannula. Similarly, other kinds of external controls may be realized, such as a slider button or an electrical switch.

In some embodiments, a position sensor is firmly attached to the internal wall of the cannula of the trocar at a distal end of the cannula. Sensor wiring, providing location data from the sensor, is passed from the sensor, through the camera control guide, and to a processor that provides the physician with location data for the trocar distal end to, for example, register a captured image from the movable (e.g., slidable) camera with a reference medical image (e.g., an MRI image).

By optimizing visual image acquisition using a position-adjustable (i.e., movable) camera of a trocar, the disclosed technique may enable improved outcomes of minimally invasive medical procedures.

### System Description

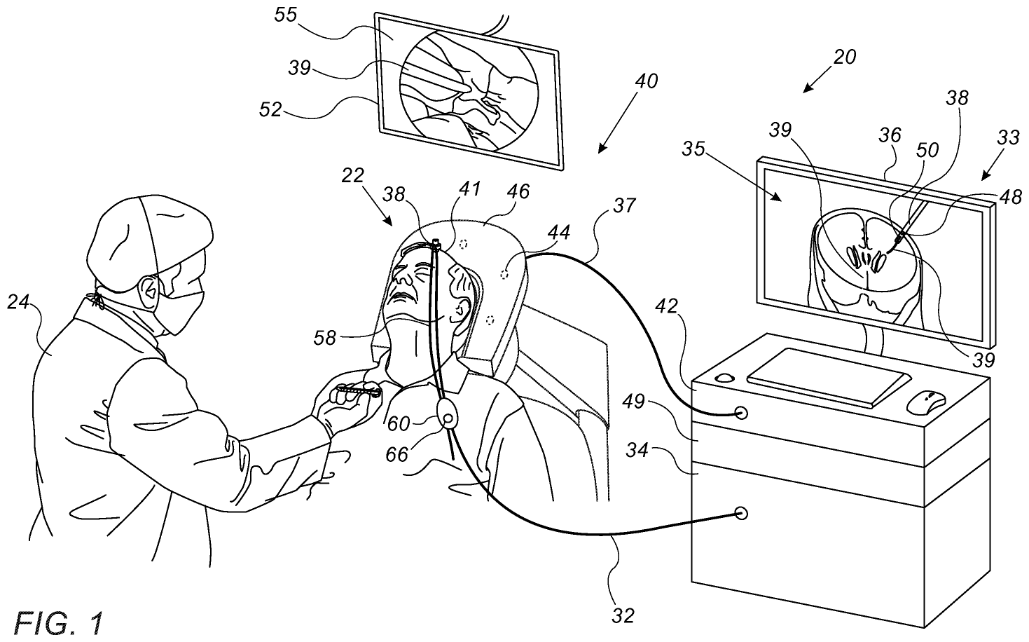

FIG. 1 is a schematic, pictorial illustration of a brain procedure using a surgical apparatus 28 comprising a trocar 38 comprising a slidable camera 48 and a position sensor 50, in accordance with an embodiment of the present invention. In some embodiments, a brain diagnostics and treatment system 20, which comprises surgical apparatus 28, is configured to carry out a brain procedure, such as treating an infection from brain tissue of a patient 22. In the shown embodiment, trocar 38 is used to penetrate the skull so that a physician 24 can insert a probe 39 into a head 41 of patient 22 to access brain tissue. Subsequently, probe 39 may be operated using the trocar-attached slidable camera 48 and magnetic position sensor 50. Typically, treating probe 39 may be operated by a second physician (not shown).

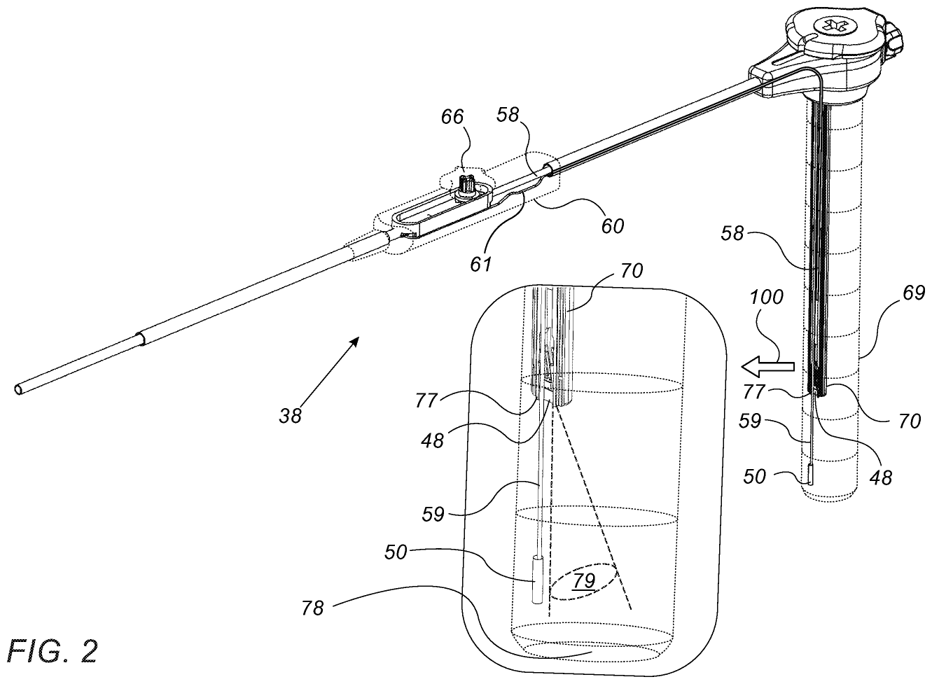

In the shown embodiment, a slide control handle 60 includes a rotatable camera-position control knob 66 located on a slide control to move a camera control guide 58 (i.e., movable element 58). Guide 58 enters a proximal end of trocar 38 and is coupled on its distal end to camera 48. By rotating knob 66, physician 24 can slide camera 48 inside trocar 38 to adjust a position of slidable camera 48 for a best view, as further described in FIG. 2.

Slide control handle 60 may further include wiring to sensor 50 and may include additional control elements to assist physician 24 to perform the procedure, such as command buttons to capture an image from camera 48 and to register the image with a reference medical image.

System 20 comprises a magnetic position-tracking system, which is configured to track a position of sensor 50 in the brain. The magnetic position-tracking system comprises a location pad 40, which comprises field generators 44 fixed on a frame 46. In the exemplary configuration shown in FIG. 1, pad 40 comprises five field generators 44, but may alternatively comprise any other suitable number of generators 44. Pad 40 further comprises a pillow (not shown) placed under head 41 of patient 22, such that generators 44 are located at fixed, known positions external to head 41. The position sensor generates position signals in response to sensing external magnetic fields generated by field generators 44, thereby enabling a processor 34 to estimate the position of sensor 50 and therefore a position of a distal edge of trocar 38 inside the head of patient 22.

This technique of position sensing is implemented in various medical applications, for example, in the CARTO™ system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1, which prior applications are hereby incorporated by reference in their entirety herein into this application as if set forth in full.

In some embodiments, system 20 comprises a console 33, which comprises a memory 49, and a driver circuit 42 configured to drive field generators 44, via a cable 37, with suitable signals so as to generate magnetic fields in a predefined working volume in space around head 41.

Processor 34 is typically a general-purpose computer, with suitable front end and interface circuits for receiving images from camera 48 and signals from position sensor 50 via a cable 32, and for controlling other components of system 20 described herein.

In some embodiments, processor 34 is configured to register an image produced by camera 48 with a medical image, such as an MRI image. Processor 34 may further register the position of the distal end that is estimated using position sensor 50. Processor 34 is able to register a visual image by estimating a position of a distal edge of trocar 38 using position sensor 50. Processor 34 is configured to register the camera image and the reference medical image in the coordinate system of the magnetic position-tracking system and/or in a coordinate system of the reference medical image.

In some embodiments, system 20 comprises a video display 52 that shows an image 55 taken by sliding camera 48. In the shown image, a distal end of treating probe 39 can be seen engaging brain tissue.

In some embodiments, processor 34 is configured to receive, via an interface (not shown), one or more anatomical images, such as reference MRI images depicting two-dimensional (2D) slices of head 41. Processor 34 is configured to select one or more slices from the MRI images, register it with a real-time camera image, such as image 55, to produce a combined image, such as an image 35, and display the selected combined slice to physician 24 on user display 36. In the example of FIG. 1, combined image 35 depicts a sectional coronal view of anterior brain tissue of patient 22.

Console 33 further comprises input devices, such as a keyboard and a mouse, for controlling the operation of the console, and a user display 36, which is configured to display the data (e.g., images) received from processor 34 and/or to display inputs inserted by a user using the input devices (e.g., by physician 24).

FIG. 1 shows only elements related to the disclosed techniques for the sake of simplicity and clarity. System 20 typically comprises additional or alternative modules and elements that are not directly related to the disclosed techniques, and thus are intentionally omitted from FIG. 1 and from the corresponding description. Alternative embodiments are possible to move the camera, such as using a slider button on handle 60 instead of a rotatable knob.

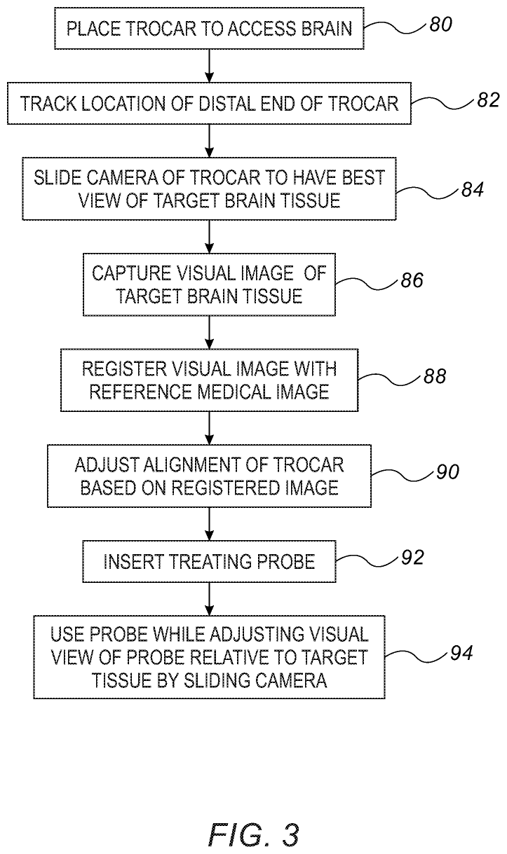

Processor 34 may be programmed in software to carry out the functions that are used by the system, and to store data in memory 49 to be processed or otherwise used by the software. The software may be downloaded to the processor in electronic form, over a network, for example, or it may be provided on non-transitory tangible media, such as optical, magnetic or electronic memory media. Alternatively, some or all of the functions of processor 34 may be carried out by dedicated or programmable digital hardware components. In particular, processor 34 runs a dedicated algorithm as disclosed herein, including in FIG. 3, that enables processor 34 to perform the disclosed steps, as further described below.

### Trocar with Slidable Camera and Built-in Position Sensor

FIG. 2 is a schematic, pictorial illustration of the trocar 38 applied in the brain procedure of FIG. 1, in accordance with an embodiment of the present invention. As seen, trocar 38 includes a channel 70 inside cannula 69, which provides a track in which to slide camera control guide 58. Camera 48 is coupled to guide 58, for example, via a sliding adapter 77 on which the camera is mounted, so that the camera can slide distally (e.g., down) or proximally (e.g., up) inside cannula 69.

To slide camera control guide 58 distally, the physician rotates control knob 66 on external control handle 60 in a first direction. The rotation of knob 66 moves camera control guide 58 forward, which is translated (e.g., by the approximately right angle turning of flexible guide 58) into distal motion of camera 48 inside cannula 69. By rotating knob 66 in the other direction, the physician pulls camera control guide 58 backwards, which is translated into proximal motion of camera 48 in cannula 69.

As further seen, magnetic sensor 50 is fixed to an inner wall of cannula 69, and its wiring 59 is routed (61) with the wiring of camera 48 to control handle 60, from which the wiring is routed to the console via cable 32 of FIG. 1.

A zoom-in (100) on a distal end of cannula 69 shows that camera 48 is mounted in a tilted configuration so as to have a central distal viewing direction pointing at a center of a distal opening 78 of cannula 69. At the same time, sensor 50 is sufficiently thin to be attached to the cannula wall, and therefore does not obstruct the field of view 79 of camera 48.

The configuration of trocar 38 in FIG. 2 is depicted by way of example for the sake of conceptual clarity. In other embodiments, additional elements may be included, such as additional ports in trocar 38 to insert medical tools to the target brain location.

FIG. 3 is a flow chart that schematically illustrates a method and algorithm for achieving a best visual image from slidable camera 48 of FIG. 1 and registering the image with a reference medical image, in accordance with an embodiment of the present invention. The process begins when physician 24 places trocar 38 to access the brain, at a trocar placement step 80.

Next, physician 24 operates system 20 to magnetically track, using signals from sensor 50, a location in the brain of a distal end of trocar 38, at a trocar position tracking step 82. Next, physician 24 operates apparatus 28, by rotating control knob 66, to slide camera 48 to have a best view (e.g., on display 52) of target brain tissue, at camera position adjustment step 84. In an image capturing step 86, physician captures an image by camera 48, to register with a reference medical image.

At an image registration step 88, based on the tracked position of trocar's 38 distal end (using sensor 50), processor 34 registers the captured image (by camera 48) with a respective reference medical image stored in memory 49, such as from an MRI scan, to produce combined image 35. In an embodiment, processor 34 is further configured to correct the reference medical images based on the registered images, for example, if the treatment removes brain tissue. In another embodiment, the processor is further configured to alert a user to a detected discrepancy between the visual image and the reference image due to, for example, a larger tumor size detected by camera 48 because of tumor growth since the reference image was taken.

Next, at a trocar adjustment step 90, using combined image 35, physician 24 adjusts an alignment of trocar 38, e.g., to allow best access to target brain tissue, such as an infected tissue. Physician 24 then inserts a treating probe 39, at a probe insertion step 92. Finally, at a treating step 94, physician 24 uses probe 39 to treat target tissue under visual guidance provided by slidable camera 48, whose position is adjusted by physician 24 by using knob 66 to obtain a best view of probe 39 relative to target tissue.

The example flow chart shown in FIG. 3 is chosen purely for the sake of conceptual clarity. In alternative embodiments physician 24 may perform additional steps, such as employing additional monitoring steps (e.g., fluoroscopy) to verify the successful outcome of the procedure, and/or apply irrigation to clear view for slidable camera 48.

Although the embodiments described herein mainly address brain procedures, the methods and systems described herein can also be used in other applications that require guiding a medical device in other organs, such as located in the abdomen or the chest.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A trocar for insertion into an organ of a patient, the trocar comprising:
a cannula having a longitudinal axis;
a camera fitted inside the cannula; and
a movable element, which is coupled to the camera and is configured to be moved along the longitudinal axis of the cannula and to move the camera along the cannula.

2. The trocar according to claim 1, wherein the movable element is configured to be slid in a channel formed at an inner wall of a cannula, parallel to the longitudinal axis, and wherein a distal end of the movable element is coupled to the camera.

3. The trocar according to claim 2, and comprising a slide control handle coupled to a proximal end of the movable element, the slide control handle comprising a rotatable camera-position control knob configured to be rotated and, by being rotated, to move the movable element and the camera.

4. The trocar according to claim 1, and comprising a position sensor, which is attached to an inner wall of a distal end of the cannula, and is configured to generate signals indicative of a position of the distal end in the organ.

5. The trocar according to claim 4, wherein the position sensor is a magnetic position sensor.

6. The trocar according to claim 1, wherein the camera is tilted relative to the longitudinal axis, so as to have a viewing direction that captures a distal opening of the cannula.

7. An apparatus, comprising:
a trocar for insertion into an organ of a patient, the trocar comprising:
a cannula having a longitudinal axis;
a camera fitted inside the cannula; and
a movable element, which is coupled to the camera and is configured to be moved along the longitudinal axis of the cannula and to move the camera along the cannula; and
a slide control handle coupled to a proximal end of the movable element and configured to move the movable element and the camera.

8. The apparatus according to claim 7, wherein the movable element is configured to be slid in a channel formed at an inner wall of a cannula, parallel to the longitudinal axis, wherein a distal end of the movable element is coupled to the camera, and wherein the slide control handle comprises a rotatable camera-position control knob configured to be rotated and, by being rotated, slide the movable element.

9. A system, comprising:
a trocar for insertion into an organ of a patient, the trocar comprising:
a cannula having a longitudinal axis;
a camera fitted inside the cannula;
a movable element, which is coupled to the camera and is configured to be moved along the longitudinal axis of the cannula and to move the camera along the cannula; and
a position sensor, which is attached to an inner wall of a distal end of the cannula, and is configured to generate signals indicative of a position of the distal end in the organ; and
a processor, which is configured to:
using the signals generated by the position sensor, estimate the position of the distal end of the trocar in the organ.

10. The system according to claim 9, wherein the processor is further configured to:
based on the estimated position, register an image acquired by the camera with a reference medical image; and
present the image acquired by the camera and the reference medical image, registered with one another, to a user.

11. The system according to claim 9, wherein the position sensor is a magnetic position sensor.

12. A method, comprising:
inserting a trocar into an organ of a patient, the trocar comprising:
a cannula having a longitudinal axis;
a camera fitted inside the cannula; and
a movable element, which is coupled to the camera and is configured to be moved along the longitudinal axis of the cannula and to move the camera along the cannula; and
moving the movable element and the camera using a slide control handle coupled to a proximal end of the movable element.

13. The method according to claim 12, wherein moving the movable element comprises rotating a camera-position control knob coupled to the slide control handle, so as to slide the movable element in a channel formed at an inner wall of a cannula, parallel to the longitudinal axis.

14. A method, comprising:
inserting a trocar into an organ of a patient, the trocar comprising:
a cannula having a longitudinal axis;
a camera fitted inside the cannula;
a movable element, which is coupled to the camera and is configured to be moved along the longitudinal axis of the cannula and to move the camera along the cannula; and
a position sensor, which is attached to an inner wall of a distal end of the cannula without obstructing a field of view of the camera, and is configured to generate signals indicative of a position of the distal end in the organ; and
using the signals generated by the position sensor, estimating the position of the distal end of the trocar in the organ.

15. The method according to claim 14, and comprising:
based on the estimated position, registering an image acquired by the camera with a reference medical image; and
presenting the image acquired by the camera and the reference medical image, registered with one another, to a user.

16. The method according to claim 14, wherein the position sensor is a magnetic position sensor.
